(19) United States
(12) Patent Application Publication
(10) Pub. No.: US 2017/0182242 A1
(43) Pub. Date: Jun. 29, 2017

(54) Wearable Automatic Injection Device and Related Methods of Use

(71) Applicant: AbbVie Inc., North Chicago, IL (US)

(72) Inventors: Charles M. Galitz, Kenosha, WI (US); Kent Abrahamson, Libertyville, IL (US); Kevin P. McLennan, Chicago, IL (US)

(73) Assignee: AbbVie Inc., North Chicago, IL (US)

(21) Appl. No.: 15/384,856

(22) Filed: Dec. 20, 2016

Related U.S. Application Data
(60) Provisional application No. 62/271,283, filed on Dec. 27, 2015.
(60) Provisional application No. 62/352,362, filed on Jun. 20, 2016.

(51) Int. Cl. A61M 5/142; B65B 7/28; A61M 5/145; B65B 3/00; A61M 5/20; A61M 5/32

(52) U.S. Cl. CPC A61M 5/14244; A61M 5/20; A61M 5/3287; A61M 5/145; B65B 3/006; B65B 7/28; A61M 2005/14506

(57) ABSTRACT

Exemplary embodiments provide wearable automatic injection devices for providing an injection of a therapeutic agent into a patient. The wearable automatic injection device includes a housing having a patient contact portion securable to the patient, an injection needle for insertion into the patient, and a prefilled syringe assembly for holding the therapeutic agent. The prefilled syringe assembly includes a distal stopper and a proximal stopper penetrated by a penetrating needle. The penetrating needle is in fluid communication with the patient injection needle.

### CROSS REFERENCE TO RELATED APPLICATIONS

The present application claims priority to U.S. Provisional Application No. 62/271,283, filed Dec. 27, 2015, and U.S. Provisional Application No. 62/352,362, filed Jun. 20, 2016, both of which are incorporated herein in their entirety.

### TECHNICAL FIELD

This disclosure relates generally to injection devices for use in delivering therapeutic fluids to a patient, and more particularly to wearable automatic injection devices and methods of using wearable automatic injection devices.

### BACKGROUND

Automatic injection devices offer an alternative to manually-operated syringes for delivering therapeutic agents into patients' bodies and allowing patients to self-administer injections. Automatic injection devices have been used to deliver medications under emergency conditions, for example, to administer epinephrine to counteract the effects of a severe allergic reaction. Automatic injection devices have also been described for use in administering anti-arrhythmic medications and selective thrombolytic agents during a heart attack (See, e.g., U.S. Pat. Nos. 3,910,260; 4,004,577; 4,689,042; 4,755,169; and 4,795,433). Various types of automatic injection devices are also described in, for example, U.S. Pat. Nos. 3,941,130; 4,261,358; 5,085,642; 5,092,843; 5,102,393; 5,267,963; 6,149,626; 6,270,479; 6,371,939; and 9,180,244 and International Patent Publication No. WO/2008/005315; each of which is incorporated by reference herein in its entirety.

Conventionally, an automatic injection device houses a syringe and, when operated, causes the syringe to move forwardly and a needle to project from the housing so that a therapeutic agent contained in the syringe is ejected into a patient's skin. An automatic injection device typically includes a bung disposed within the syringe that when actuated, moves within the syringe to expel the therapeutic agent from the syringe and into the patient's skin. During operation of conventional automatic injection devices, a user actuates a firing button to cause the syringe needle to penetrate the skin and move the syringe bung within the syringe to expel the therapeutic agent into the patient's skin.

Conventional automatic injection device users may experience anxiety, pain, discomfort and frustration preparing for, actuating, upon skin penetration, and during injection, resulting in unacceptably high treatment abortion rates, non-compliance and non-persistence. Wearable automatic injection devices, such as Wearable Bolus injectors (WBI) or On-Body Delivery Systems (OBDS) for subcutaneous uses are preferred by many patients over conventional automatic injection devices, because patients associate subcutaneous injection and delivery with a WBI or OBDS as less painful, simpler, easier to learn, and generally less threatening and more comfortable. However, conventional WBI and OBDS devices are expensive, require custom filling and are bulky in size.

Accordingly, there remains a need for further improvement of known automatic injection devices. For example, it may be desirable to provide a wearable automatic injection device that can be configured for use with a variety of prefilled therapeutic agent containers, such as syringes, cartridges, vial or ampules. It may also be desirable to configure such devices by forming sterile barriers in the wearable automatic injection device while being easy to manufacture and use.

### SUMMARY

The purpose and advantages of the disclosed subject matter will be set forth in and apparent from the description that follows, as well as will be learned by practice of the disclosed subject matter. Additional advantages of the disclosed subject matter will be realized and attained by the methods and systems particularly pointed out in the written description and claims hereof, as well as from the appended drawings.

To achieve these and other advantages and in accordance with the purpose of the disclosed subject matter, as embodied and broadly described, exemplary embodiments of the disclosed subject matter provide wearable automatic injection devices that may adhere to the skin or clothing of a patient and deliver a therapeutic agent into the patient's body by subcutaneous injection at slow, controlled injection rates, e.g., in a single slow bolus. Exemplary embodiments provide methods of assembling exemplary wearable automatic injection devices. Exemplary embodiments also provide methods of using wearable automatic injection devices worn by a patient for slow, controlled therapeutic agent delivery. Exemplary wearable automatic injection devices reduce or eliminate a burning sensation often felt or perceived by patients who use a conventional automatic injection device. Exemplary wearable automatic injection devices maintain the sterility of the therapeutic agent container (e.g., syringe, cartridge, vial, ampoule), are easy to use, pre-fill capable, easy to manufacture, and/or do not require aseptic assembly. The wearable automatic injection devices provided by exemplary embodiments may deliver any therapeutic agent including, but not limited to, a biologic drug, such as, for example, an antibody, insulin, etc.

In accordance with an exemplary embodiment, a wearable automatic injection device is provided for providing an injection of a therapeutic agent into a patient. The device includes a housing comprising a contact portion securable to the patient, and an injection assembly moveably disposed in the housing holding an injection needle for insertion into the patient. The injection assembly is movable between a retracted position in which the injection needle does not protrude outside the housing and an extended position in which the injection needle protrudes outside the housing. The device includes a prefilled container assembly disposed in the housing for holding the therapeutic agent, the prefilled container assembly may include a barrel with a sealed distal end, the barrel may have a first stopper, a second stopper spaced from and movable relative to the first stopper, and a dispensing member coupled to a distal portion of the second stopper and configured to engage the first stopper. The dispensing member may be in fluid communication with the injection needle.

In some examples, the wearable automatic injection device may include all or a combination of the following features: a fluid line coupled to and extending between the patient injection needle and the penetrating member, the fluid line may be movable with the injection assembly, a fluid drive mechanism may be configured to urge the second stopper in a distal direction toward the first stopper, the fluid drive mechanism may be selected from the group consisting of a: hydraulic mechanism, pneumatic mechanism, spring motor or clock spring mechanism, cam actuator, and compression spring, the housing may include a top surface comprising a viewing window radially aligned with the contents of the barrel, the housing may have a bottom surface comprising an adhesive surface defining the contact portion, and the bottom surface may have an aperture therethrough, the patient injection needle extending through the aperture when the injection assembly is in the extended position.

In accordance with another exemplary embodiment, a prefilled container assembly is provided having a container including a barrel having a sealed distal end and a proximal end, a first stopper and a second stopper spaced from the first stopper toward the proximal end and movable relative to the first stopper; a dispensing member coupled to a distal portion of the second stopper and configured to engage the first stopper; and a fluid tube in fluid communication with the dispensing member and an injection needle.

In some examples, the container may include a syringe or a cartridge.

In accordance with another exemplary embodiment, a method is provided for injecting a therapeutic agent into a patient. The method includes providing a container prefilled with the therapeutic agent and a first stopper, urging a second stopper having a dispensing needle extending therefrom into engagement with the first stopper, and delivering the therapeutic agent to the patient via an injection needle in fluid communication with the second stopper.

In accordance with another exemplary embodiment, a method is provided for assembling a prefilled container and injection assembly including sealing a distal end of a container; inserting a fluid through a proximal end of the container; inserting a first stopper through the proximal end of the container to seal the fluid therein; inserting a second stopper through the proximal end of the container, the second stopper having a dispensing member extending therefrom toward the first stopper; joining the second stopper to a first end of a tubing member extending through the proximal end of the container; and joining an opposing end of the tubing member to an injection needle.

In accordance with another exemplary embodiment, a wearable automatic injection device is provided for injection of a therapeutic agent into a patient, the wearable automatic injection device comprises a housing comprising a contact portion configured to secure to the patient; an injection assembly moveably disposed in the housing, the injection assembly including an injection needle to be inserted into the patient, the injection assembly movable between a retracted position in which the injection needle does not protrude outside the housing and an extended position in which the injection needle protrudes outside the housing; a prefilled container assembly provided in the housing, the prefilled container assembly comprising a container with a sealed distal end, a first stopper spaced from the distal end and defining a fluid reservoir therebetween to hold a mixing fluid, a second stopper spaced from the first stopper toward the proximal end and movable relative to the first stopper, and a dispensing member extending from the second stopper toward the first stopper and configured to engage the first stopper, the dispensing member in fluid communication with the injection needle via a fluid line; and a mixing chamber containing the therapeutic agent, the mixing chamber disposed between and in fluid communication with the prefilled container assembly and the injection needle via the fluid line.

In accordance with another exemplary embodiment, a prefilled container assembly is provided, the prefilled container assembly comprises a barrel having a sealed distal end and a proximal end; a first stopper spaced from the distal end and defining a fluid reservoir therebetween; a second stopper spaced from the first stopper toward the proximal end; a dispensing member joined to the second stopper and configured to engage the first stopper; and a fluid tube in fluid communication with the dispensing member and an injection needle.

In some examples, the prefilled container assembly may include all or a combination of the following features: the prefilled container may include a fluid therapeutic agent in the fluid reservoir. the dispensing member may be configured to engage the first stopper and define a channel therethrough to allow the fluid therapeutic agent to move from the fluid reservoir to the fluid tube through first and second stoppers, the distal end of the barrel may have a diameter less than a diameter of the proximal end of barrel, the second stopper may be configured to urge the first stopper toward the distal end of the barrel, the injection needle may be configured to penetrate the skin of a patient and deliver the fluid therapeutic agent from the fluid reservoir to patient, the fluid therapeutic agent may include a biological agent.

It is to be understood that both the foregoing general description and the following detailed description are exemplary and explanatory only and are not restrictive of the invention, as claimed.

The accompanying drawings, which are incorporated in and constitute a part of this specification, illustrate several embodiments of the disclosure and together with the description, serve to explain the principles of the invention.

### BRIEF DESCRIPTION OF THE DRAWINGS

FIG. 1 is a front view of a combined prefilled container and injection assembly according to an illustrative embodiment of the disclosed subject matter.

FIGS. 2A-2C are perspective views of various components of the prefilled container and injection assembly of FIG. 1 according to an illustrative embodiment of the disclosed subject matter.

FIG. 3 is a perspective view of a prefilled container and injection assembly according to another illustrative embodiment of the disclosed subject matter.

FIG. 4 is a perspective view of various components of the prefilled container and injection assembly of FIG. 3 according to an illustrative embodiment of the disclosed subject matter.

FIG. 5A is a top-left perspective view of a wearable automatic injection device according to an illustrative embodiment of the disclosed subject matter.

FIG. 5B is a top-left perspective view of the wearable automatic injection device of FIG. 5A, with the housing being transparent, for purpose of illustration, to show various internal components of the device according to an illustrative embodiment of the disclosed subject matter.

FIG. 5C is a bottom-left perspective view of the wearable automatic injection device of FIG. 5A according to an illustrative embodiment of the disclosed subject matter.

FIG. 6 is a schematic view of a hydraulic actuation mechanism of a wearable automatic device according to an illustrative embodiment of the disclosed subject matter.

FIG. 7A is a schematic view of a spring motor actuation mechanism of a wearable automatic device according to an illustrative embodiment of the disclosed subject matter.

FIG. 7B is a schematic view of gear train components of the spring motor actuation mechanism of FIG. 7A.

FIG. 8 is a schematic view of a cam spring actuation mechanism of a wearable automatic device according to an illustrative embodiment of the disclosed subject matter.

FIG. 9 is a schematic view of a compression spring actuation mechanism of a wearable automatic device according to an illustrative embodiment of the disclosed subject matter.

FIG. 10 is a schematic view of a patient needle retraction mechanism of a wearable automatic device according to an illustrative embodiment of the disclosed subject matter.

FIG. 11 is a flow diagram illustrating a sterilization procedure for assembling a wearable automatic injection device according to an illustrative embodiment of the disclosed subject matter.

FIG. 12 is a flow diagram illustrating another sterilization procedure for assembling a wearable automatic injection device according to an illustrative embodiment of the disclosed subject matter.

FIG. 13A is a flow diagram illustrating another sterilization procedure for assembling a wearable automatic injection device according to an illustrative embodiment of the disclosed subject matter.

FIG. 13B is a flow diagram illustrating another sterilization procedure for assembling a wearable automatic injection device according to an illustrative embodiment of the disclosed subject matter.

FIG. 14A is a perspective view of pain reduction components of a wearable automatic injection device according to an illustrative embodiment of the disclosed subject matter.

FIG. 14B is a cross-sectional view of the pain reduction components taken along line B-B of FIG. 14A according to an illustrative embodiment of the disclosed subject matter.

FIG. 15A is a schematic view of a flow regulator, operating at a first temperature, for use in a wearable automatic injection device according to an illustrative embodiment of the disclosed subject matter.

FIG. 15B is a schematic view of the flow regulator of FIG. 15A operating at a second temperature.

FIG. 16A is an illustration of another flow regulator, operating at a first temperature, for use in a wearable automatic injection device according to an illustrative embodiment of the disclosed subject matter.

FIG. 16B is a schematic view of the flow regulator of FIG. 16A operating at a second temperature.

FIG. 17 is a schematic view of an audible feedback mechanism for use in a wearable automatic injection device according to an illustrative embodiment of the disclosed subject matter.

FIGS. 18A and 18B are schematic views of a visual feedback mechanism for use in a wearable automatic injection device according to an illustrative embodiment of the disclosed subject matter.

FIG. 19 is schematic view of a reconstitution assembly for use in a wearable automatic injection device according to an illustrative embodiment of the disclosed subject matter.

### DETAILED DESCRIPTION

Reference will now be made in detail to the present embodiments (exemplary embodiments) of the disclosure, examples of which are illustrated in the accompanying drawings. The structure and corresponding method of operation of the disclosed subject matter will be described in conjunction with the detailed description of the system. Wherever possible, the same reference numbers will be used throughout the drawings to refer to the same or like parts.

The apparatus and methods presented herein can be used for injecting any of a variety of suitable therapeutic agents or substances, such as a drug, into a patient. As used herein, an “automatic injection device” or “autoinjector” (used interchangeably herein) is intended to refer generally to a device that enables an individual (also referred to herein as a user or a patient) to self-administer a dosage of a liquid substance, such as a therapeutic agent, including a formulation in liquid form, wherein the device differs from a standard prefilled therapeutic agent container by the inclusion of a mechanism for automatically delivering the medication to the individual by injection when the mechanism is activated.

Subcutaneous injection is a typical mode of therapeutic agent delivery and involves administering a bolus of a therapeutic agent into a patient. Subcutaneous injections are highly effective in administering various therapeutic agents including insulin, vaccines, and drugs. Automatic injection devices offer an alternative to a manual injection device, such as a syringe, for delivering a therapeutic agent and allow patients to self-administer subcutaneous injections of therapeutic agents. Conventional automatic injection devices include hand held automatic injection devices and patch pumps, which are self-adhesive, patient-mounted autoinjectors. In use, a patch pump containing a therapeutic agent is mounted onto the skin or clothing of a patient and triggered to inject the therapeutic agent into the patient. Conventional patch pumps are typically filled by a patient prior to use. In addition, certain conventional patch pumps have an exposed needle inside the pump, and thus require secondary sterile packaging to maintain sterility.

There can be a correlation between the injection rate of certain therapeutic agents and the pain perceived by a patient upon injection of the therapeutic agents or agents. Some therapeutic agents can cause pain, e.g., a burning or stinging sensation, when injected rapidly into the patient. The pain sensation may be the result of a physiological response of the patient's skin to the subcutaneous injection of a therapeutic agent. Large volumes of any therapeutic agent, greater than one milliliter, may also cause pain when injected into the skin. Antibodies, and portions thereof, are exemplary therapeutic agents that are least painful when delivered at slow injection rates. Commercially available prefilled patch pumps can fail to effectively address the discomfort associated with fast injection rates of hand held automatic injection devices that are prefilled, or configured for delivery of a therapeutic agent in seconds or a few minutes.

Exemplary embodiments are described below with reference to certain illustrative embodiments. While exemplary embodiments are described with respect to using a wearable automatic injection device to provide an injection of a dose of a liquid medication, one of ordinary skill in the art will recognize that exemplary embodiments are not limited to the illustrative embodiments and that exemplary automatic injection devices may be used to inject any suitable substance into a patient. In addition, components of exemplary automatic injection devices and methods of making and using exemplary automatic injection devices are not limited to the illustrative embodiments described below.

The 1 mL prefilled glass standard syringe with pre-staked injection needle and needle cover has become a standard delivery system format for subcutaneously injected biotechnology drug products. The availability of a prefilled 1 mL syringe that ships to the user (patient, caregiver, or health care professional—HCP), allows for a relatively straightforward administration of the drug solution by users outside the HCP environment. Establishing a prefilled 1 mL glass syringe as primary package material as product presentation for an FDA and/or EMA approved drug can involve significant financial, regulatory and development time investments, which typically amounts to years of validation and qualification of drug product stability and fill-finish processes.

Additionally, certain prefilled syringes and disposable autoinjectors can cause patients to experience anxiety, pain, discomfort and frustration during injection, which can result in increased treatment abortion rates, non-compliance and non-persistence. Wearable automatic injection devices, such as Wearable Bolus Injectors (WBI) or On-Body Delivery Systems (OBDS) can be preferred by many patients over prefilled syringes, because patients associate the subcutaneous injection and delivery with a WBI or OBDS as less painful, simpler, easier to learn, and generally less threatening and more comfortable. The present subject matter thus provides the dosing experience patients perceive and experience with a wearable automatic injection device using a prefilled syringe as primary package material.

Certain embodiments described herein use a prefilled syringe or syringe barrel/cartridge as primary package material while at the same time providing a wearable automatic injection device to the user by forming sterile barriers in wearable bolus injector form factors by design and assembly of prefilled syringe primary packaging, fluid path, and needle insertion assembly and stopper penetration mechanisms. While the present disclosed subject matter is described with respect to a wearable automatic injection device to provide a subcutaneous injection of a dose from a prefilled syringe, one skilled in the art will recognize that the disclosed subject matter is not limited to the illustrative embodiment, and that the injection device can be used to inject a substance from any suitable container, including but not limited to prefilled syringes, cartridges, vials or ampules.

A syringe assembly of exemplary wearable automatic injection devices, such as WBI or OBDS devices, may contain a dose of any suitable therapeutic agent, for example, an antibody, a cytokine, a vaccine, a fusion protein or a growth factor. In a preferred embodiment, therapeutic agent is a TNFα inhibitor (e.g., an anti-TNF antibody or an antigen-binding portion thereof, a TNF fusion protein, or a recombinant TNF binding protein), such as adalimumab (HUMIRA®). Another particularly preferred therapeutic agent for use in the wearable automatic injection device is an isolated human antibody that dissociates from human TNFα with a K_{d} of 1×10⁻⁸ M or less and a K_{off} rate constant of 1×10⁻³ s⁻¹ or less, both determined by surface plasmon resonance, and neutralizes human TNFα cytotoxicity in a standard in vitro L929 assay with an IC₅₀ of 1×10⁻⁷M or less. In one example, the wearable automatic injection device, including uses and compositions thereof, comprises a dose of a TNFα inhibitor. In one example, the anti-TNFα antibody, or antigen-binding portion thereof, is a chimeric antibody, a humanized antibody, a human antibody, and a multivalent antibody. In one example, the anti-TNFα antibody is an isolated human antibody, or antigen-binding portion thereof and dissociates from human TNFα with a K_{off} rate constant of 1×10⁻³ s⁻¹ or less, as determined by surface plasmon resonance.

In an exemplary embodiment, the human TNFα antibody or antigen-binding portion thereof may be adalimumab or golimumab. In another example, the human TNFα antibody or antigen-binding may be ABT-122 dual variable domain immunoglobulin. In another exemplary embodiment, the therapeutic agent may include a monoclonal antibody targeting interleukin 23A (IL-23A).

Exemplary embodiments provide wearable automatic injection devices that may adhere to the skin or clothing of the patient and deliver a therapeutic agent into patient by subcutaneous injection at slow, controlled injection rates, e.g., in a single slow bolus. The controlled injection rates achieved by exemplary devices in combination with the dosing experience of a wearable bolus injector may minimize the pain sensation associated with a volume of a therapeutic agent entering into the patent's tissue. Exemplary time durations for slow delivery achieved by exemplary devices may range from about 10 seconds to about 10 minutes and may depend on the injection volume, but are not limited to this exemplary range. Exemplary volumes of therapeutic agent deliverable by exemplary devices may range from about 0.4 milliliters to about 2.25 milliliter, but are not limited to this exemplary range. In addition, exemplary devices may advantageously minimize inflections in the delivery profile against time of the therapeutic agent. Exemplary viscosities of therapeutic agent deliverable by exemplary devices may range from about 1 cPs to about 50 cPs, but are not limited to this exemplary range. In addition, exemplary devices may advantageously minimize inflections in the delivery profile against time of the therapeutic agent.

Exemplary embodiments reduce or minimize the size envelope of exemplary wearable automatic injection devices, and provide scalable solutions with configurable delivery times and delivery profiles that may be used for a range of therapeutic agent viscosities.

Exemplary embodiments may use prefilled containers, such as syringes, cartridges, vials or ampules, with industry standard configurations of 1 mL, 1.5 mL and 2.25 mL. Scalable solutions with configurable delivery times and delivery maybe achieved by using 10 mL and 20 mL prefilled syringes.

Exemplary embodiments of the wearable automatic injection device may use prefilled syringes with staked needles covered by rubber needle shields, or rubber and rigid needle shield combinations as sterile barriers.

Exemplary embodiments of the wearable automatic injection device may use prefilled syringes where the step of needle staking and needle covering with needle shields to provide a sterile barrier has been skipped and omitted during the manufacturing process of the syringe.

Other exemplary embodiments of the wearable automatic injection device may use prefilled syringes where the manufacturing steps of needle staking and needle covering with needle shields and forming the cone or conical portion at the tip of the needle have been skipped and omitted.

Exemplary embodiments provide wearable automatic injection devices that deliver a therapeutic agent into a patient by subcutaneous injection at slow, controlled injection rates, e.g., in a single slow bolus. Exemplary embodiments may also provide methods of using the wearable automatic injection devices for slow, controlled therapeutic agent delivery. The wearable automatic injection devices provided by exemplary embodiments are pre-fillable prior to delivery to the patient; maintain sterility of the therapeutic agent and all subcutaneous contact surfaces (i.e., an injection needle) to avoid the need for aseptic assembly and address the perceived patient discomfort due to injection by conventional hand held automatic injection devices. Exemplary wearable automatic injection devices are disposable, easy to use, pre-fill capable, and may substantially or completely eliminate injection discomfort and/or injection anxiety often experienced by a patient that uses a prefilled syringe or a wearable automatic injection device that a patient needs to fill and self-assemble before injection of the drug occurs. The wearable automatic injection devices provided by exemplary embodiments can be used to deliver any therapeutic agent that may be delivered subcutaneously including, but not limited to, an antibody or insulin, etc.

The wearable automatic injection device of the exemplary embodiments may include a “therapeutically effective amount” or a “prophylactically effective amount” of a therapeutic composition such as an antibody or antibody portion. A “therapeutically effective amount” refers to an amount effective, at dosages and for periods of time necessary, to achieve the desired therapeutic result. A therapeutically effective amount of an antibody, antibody portion, or other TNFα inhibitor may vary according to factors such as the disease state, age, sex, and weight of the patient, and the ability of the antibody, antibody portion, or other TNFα inhibitor to elicit a desired response in the patient. A therapeutically effective amount can also be one in which any toxic or detrimental effects of the antibody, antibody portion, or other TNFα inhibitor are outweighed by the therapeutically beneficial effects. A “prophylactically effective amount” refers to an amount effective, at dosages and for periods of time necessary, to achieve the desired prophylactic result. Typically, since a prophylactic dose is used in patients prior to or at an earlier stage of disease, the prophylactically effective amount will be less than the therapeutically effective amount.

The terms “substance” and “therapeutic agent” refer to any type of drug, biologically active agent, biological substance, chemical substance or biochemical substance that is capable of being administered in a therapeutically effective amount to a patient employing exemplary automatic injection devices. Exemplary substances include, but are not limited to, agents in a liquid state. Such agents may include, but are not limited to, adalimumab (HUMIRA®), ABT-122 (a dual variable domain immunoglobulin targeting TNFα. IL-23A and IL-17, and proteins that are in a liquid solution, e.g., fusion proteins and enzymes.

In an exemplary wearable automatic injection device including a prefilled container assembly, a dispensing needle may be coupled directly to the barrel portion of the syringe and may be in fluid communication with the barrel portion. The term “pre-injection state” refers to a state of a wearable automatic injection device prior to the start of delivery of a therapeutic agent contained in the device. The term “injection state” refers to one or more states of a wearable automatic injection device during the delivery of a therapeutic agent contained in the device. The term “post-injection state” refers to completion of delivery of a therapeutically effective dose of a therapeutic agent contained in the device and also to removal of the device from the patient prior to completion of delivery of a therapeutically effective dose of the therapeutic agent.

The term “slow” refers to a delivery rate of a volume of a therapeutic agent. In an exemplary embodiment, a volume of about 0.1 milliliters to about 1 milliliter or more may be delivered in a delivery time period of about ten seconds to about 60 seconds. In a preferred embodiment, the delivery time period may range from about 8 seconds to about 20 seconds for up to 1 milliliter volume of therapeutic agent, from about 20 seconds to about 60 seconds for up to 1.5 mL, from about 60 seconds to about 140 seconds for up to 2.25 mL. For every 0.4 mL delivery volume added to the delivery volume of 2.25 mL delivered in about 60 seconds to 140 seconds, an additional delivery time of about 50-70 seconds may be added to the overall delivery time.

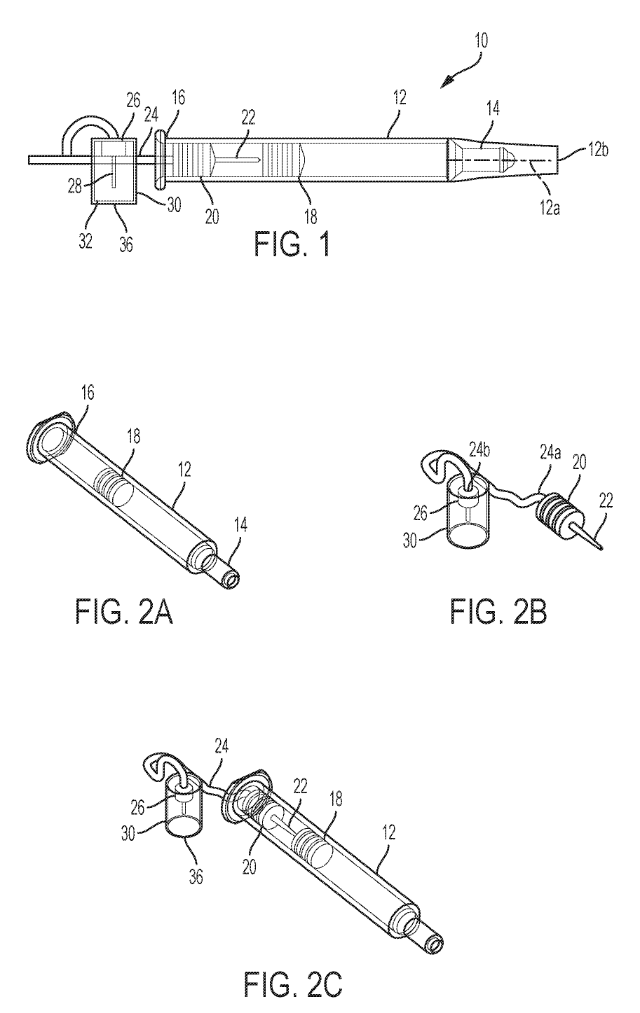

The accompanying figures, where like reference numerals refer to identical or functionally similar elements throughout the separate views, serve to further illustrate various embodiments and to explain various principles and advantages all in accordance with the disclosed subject matter. For purpose of explanation and illustration, and not limitation, exemplary embodiments of the wearable automatic injection device, and exemplary techniques for making and using the wearable automatic injection device, in accordance with the disclosed subject matter are shown in FIGS. 1-19.

Exemplary wearable automatic injection devices may employ a prefilled container assembly, which can include a prefilled container, such as a syringe or cartridge, for holding a dose of a therapeutic agent that may be delivered into a patient's body through an injection assembly. Generally, and unless otherwise noted, the term “first end” or “distal end” refers to the portion or end of the prefilled container having a sealed end to prevent the therapeutic agent from exiting therethrough. The term “second end” or “proximal end” refers to the portion or end of the prefilled container spaced from the distal end and having a channel or opening to allow the therapeutic agent to exit therethrough. As such, the prefilled container and injection assembly is configured to deliver the therapeutic agent from a proximal end of the prefilled container via a cannula disposed proximate the proximal end of the syringe, the cannula being in fluid communication with the injection assembly, which can include a patient needle. The prefilled container assembly and the injection assembly can be joined, for example and as embodied herein, via a fluid channel.

FIGS. 1 and 2A-2C illustrate views of an exemplary prefilled container assembly (FIG. 2A) and injection assembly (FIG. 2B) for use in a wearable auto-injection device in a pre-injection state. As shown in FIG. 1, a combined prefilled container and injection assembly 10 may include a syringe 12 having a barrel, a distal end 14, a proximal end 16, and also may include hypodermic needle 12a and a needle cover 12b. In some examples, as shown in FIGS. 2A-2C one or more of the hypodermic needle 12a, needle cover 12b, and/or conical portion, may be removed and the distal end 14 of the syringe may be sealed (FIG. 2A). The syringe may be manufactured using any suitable materials, such as glass (e.g. glass type 1), plastic, COP (cyclo-olefin polymer), and/or polypropylene. In some examples, the syringe may not include the hypodermic needle 12a and needle cover 12b. In some examples, the syringe may be configured to hold up to 1 mL of fluid and may be manufactured having the pre-staked the hypodermic needle 12a and cover 12b. The distal end 14 of the syringe 12 may be sealed in any suitable manner to prevent fluid in the syringe 12 from exiting the syringe from the distal end 14 of the syringe 12.

The prefilled container and injection assembly 10 may include a first and second sealing component, such as stopper, such as a distal stopper 18 and a proximal stopper 20. The proximal stopper 20 may include a dispensing member 22 extending therefrom or therethrough, which can include a dispensing needle joined to the proximal stopper 20 by overmolding, adhesive, bonding, or via mechanical fixation. The dispensing member 22 may be configured to penetrate the distal stopper 18, as described herein. The dual stoppers (distal and proximal stoppers 18, 20) may be manufactured using the same or different materials and the dispensing member 22 extending from or disposed through the proximal stopper 20 may include a tip configured to minimize coring of the proximal stopper 20. The stoppers 18 and 20 may be configured in the syringe 12 to maintain sterile barriers for maintaining the sterility of the therapeutic agent held in the syringe 12. The prefilled container and injection assembly 10 may include tubing 24 having a first end 24a in fluid communication with the proximal stopper 20 and dispensing member 22 and a second end 24b in fluid communication with a needle hub 26. The tubing 24 may be manufactured using polypropylene, PVC, silicone, Teflon or any other suitable materials. The needle hub 26 may be in fluid communication with an injection needle 28 and disposed at a proximal end of the injection needle 28, the distal end of the injection needle 28 being configured to inject the therapeutic agent in the syringe 12 to a patient. The needle hub 26 may be manufactured using any suitable material(s) such as polyester, co-polyester, or other thermoplastics. The dispensing member 22 and injection needle 28 may be manufactured using any suitable materials such as stainless steel. The dispensing needle 22 and/or injection needle 28 may have any suitable gauge/length. In some embodiments, the length of each needle 22 and 28 may be less than the gauge/length of a hypodermic needle attached to a conventional prefilled syringe for subcutaneous delivery (e.g. via an autoinjector). As shown in FIG. 1, the injection needle 28 is in a retracted pre-injection state and is enclosed in a sterile enclosure 30. The sterile enclosure may include a hole or aperture 36 configured to allow the injection needle 28 to contact the skin of a patient (e.g. on the arm, stomach, thigh, etc.) and deliver (e.g. subcutaneously) the therapeutic agent in the syringe 12. The hole 36 may be covered by a cover 32 and thereby sterility may be maintained by the cover 32, which can be configured, for example and without limitation, as a medical grade paper barrier that may be removed by the patient prior to use.

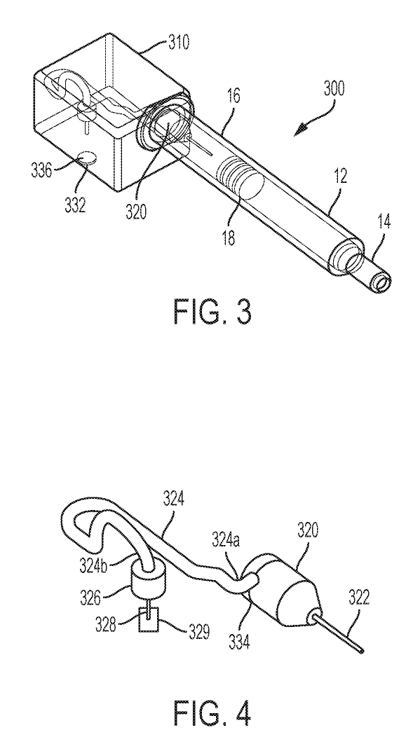

FIGS. 3 and 4 illustrate views of an exemplary prefilled container and injection assembly 300 similar to the assembly described above with reference to FIGS. 1 and 2A-2C for use in a wearable auto-injection in a pre-injection state. As shown in FIG. 3, prefilled container and injection assembly 300 may include a syringe 12 similar to the syringe described in reference to FIGS. 1 and 2A-2C having a distal end 14, a proximal end 16, and also may include hypodermic needle 12a and a needle cover 12b (shown in FIG. 1). The proximal end 16 of the syringe 12 may be enclosed in a sterile enclosure 310.

The prefilled container and injection assembly 300 may include a distal stopper 318 and a proximal stopper 320. The proximal stopper 320 may be penetrated by a dispensing needle 322 similar to dispensing member 22. The prefilled container and injection assembly 300 may include tubing 324 having a first end 24a in fluid communication with the proximal stopper 320 and disposed proximate a slot 334 formed in a portion of the proximal stopper 320 and a dispensing needle 322. Tubing 324 may include a second end 324b in fluid communication with a needle hub 326. As shown in FIGS. 3 and 4, injection needle 328 is in a retracted pre-injection state and is enclosed in the sterile enclosure 310. The sterile enclosure 310 may include a hole or aperture 336 configured to allow the injection needle 328 to contact the skin of a patient and deliver (e.g. subcutaneously) the therapeutic agent in the syringe 12. The hole 336 may be covered by a cover 332 and thereby sterility may be maintained by the cover 332, which can be configured, for example and without limitation, as a medical grade paper barrier that may be removed by the patient prior to use. In some examples, the injection needle 328 may be covered by a sterile barrier 329 which may be removed during assembly of the prefilled container and injection assembly, as described herein.

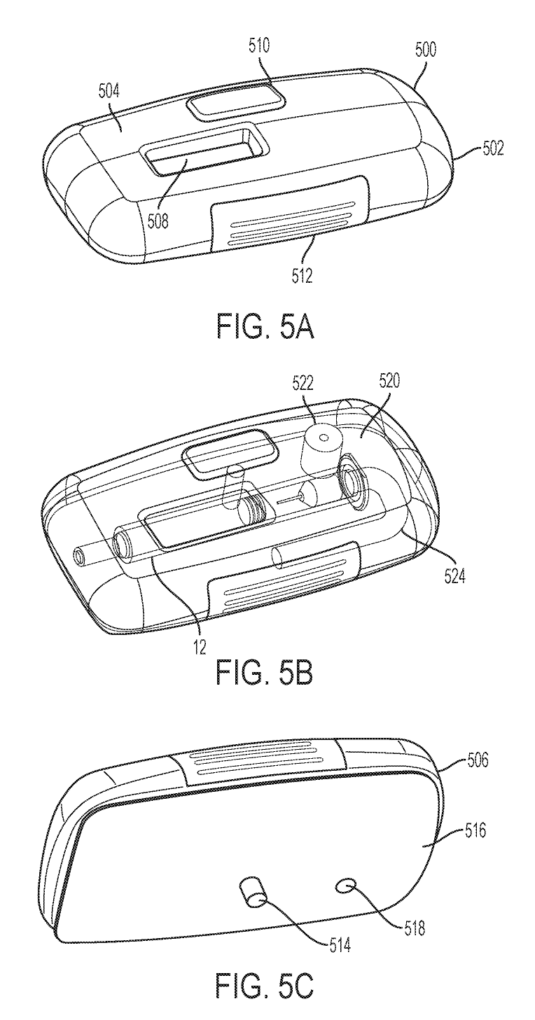

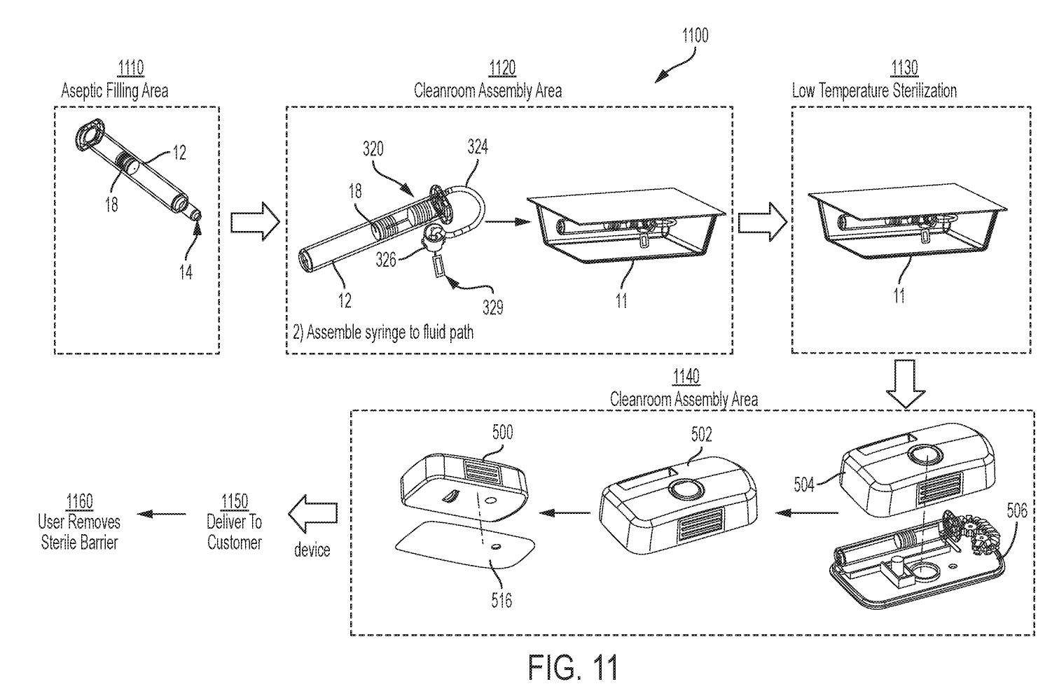

FIGS. 5A-5C together illustrate an exemplary wearable automatic injection device 500 in accordance with the disclosed subject matter. In some examples, the wearable automatic injection device 500 may be disposable/single use or may be re-used upon replacement of one or more portions of the prefilled container and injection assembly. The wearable automatic injection device 500 may be disposed in a packing container 11, such as a tub (shown in FIG. 11) to maintain sterility of the device 500 during shipping to the user. The packing container 11 may include a removable layer, which can be configured as a layer of high-density polyethylene fiber, to allow access to and removal of the device 500 from the container 11. The wearable automatic injection device 500 may include a housing 502 configured to house the prefilled container and injection assembly, such as assemblies 10 and 300, and various other components such as fluid drive mechanisms and needle retraction and insertion mechanisms, as described herein. The housing 502 includes a top surface 504 and a bottom surface 506. The housing 502 may include various features such as contours, gripping surfaces 512, one or more viewing windows 508 for viewing the contents of the syringe 12 housed in the wearable automatic injection device 500 and one or more actuators, such as actuation button 510. The actuation button 510 may be configured to allow or urge movement of the injection needle into an extended position to inject therapeutic agent from the syringe and also may actuate the fluid drive mechanism to urge the proximal stopper in a distal direction such that the dispensing needle penetrates the distal stopper to allow the flow of the therapeutic agent through the tubing and the injection needle. The actuation button may include features to prevent accidental actuation.

The wearable automatic injection device 500 may be manufactured using any suitable materials. The wearable automatic injection device 500 also may include other actuators or control features configured to control various functions of the wearable automatic injection device 500 and also may include a display (not shown) for displaying information about the wearable automatic injection device 500 and/or receiving input from the user of the wearable automatic injection device 500. As shown in FIG. 5C, the bottom surface of the housing 506 of the wearable automatic injection device 500 may include an adhesive surface 516 configured to adhere to an injection site, such as the skin of the user. The adhesive surface 516 may be double-sided and may be covered by a removable cover, such as removable/peelable paper or synthetic materials (not shown) configured to preserve the adhesiveness of the adhesive surface 516 until use. In some examples, the device 500 may be held to the injection site with one or more elastic straps in addition to or as an alternative to the adhesive surface. The elastic straps may be mechanically attached to a portion of the device 500. The wearable automatic injection device 500 may include a hole or aperture 518 for a needle, such as injection needle 28, 328 to contact the injection site. The aperture 518 may be removably covered by a sterile barrier The wearable automatic injection device 500 also may include a surface sensor 514 configured to prevent actuation of the wearable automatic injection device 500 unless contact with a patient injection site is detected. The sensor 514 may include one or more sensors configured to detect contact with the injection site, for example and without limitation, a displacement sensor, a position sensor, a pressure sensor, an optical sensor, a temperature sensor, or any other suitable sensor or combination thereof.

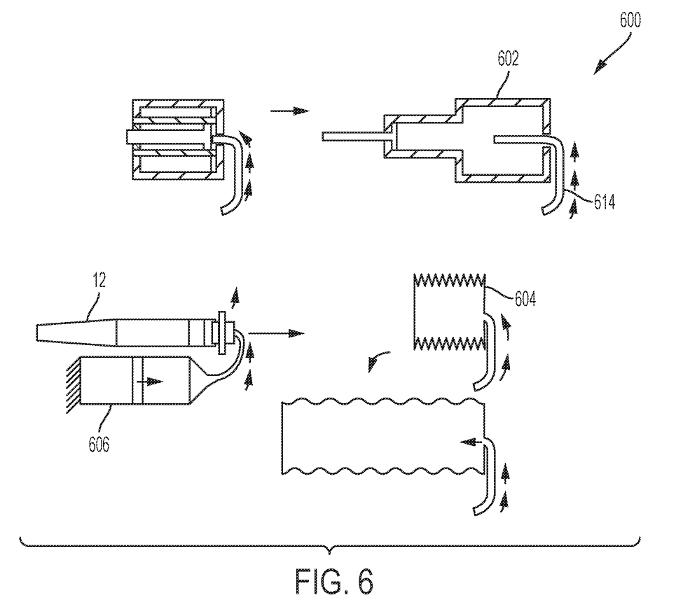

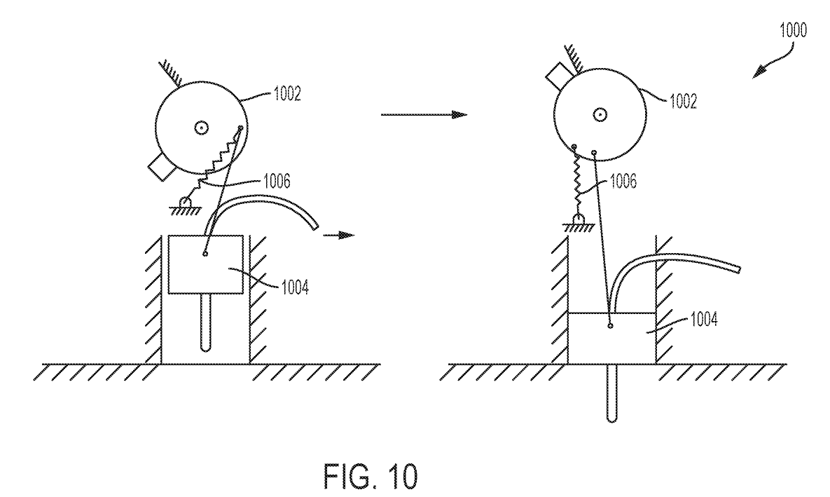

With continued reference to FIGS. 5A-5C, the wearable automatic injection device 500 includes a fluid pathway 522 from the syringe 12 to the injection needle 28, 328. Additionally, as embodied herein, the wearable automatic injection device 500 includes an injection needle 28, 328 retraction mechanism 522 (described in more detail in reference to FIG. 10) and a drive mechanism 524. The drive mechanism 524 is configured to move the proximal stopper 20, 320 in a distal direction towards distal stopper 18, 318 such that the dispensing member 22, 322 extending from the proximal stopper penetrates the distal stopper and allows therapeutic agent in the syringe 12 to flow through the dispensing member 22, 322 and tubing 24, 324 and deliver the therapeutic agent to the patient through the injection needle 28, 328. The drive mechanism 524 may thus move the proximal stopper 20, 320, and in turn the distal stopper 18, 318 in a distal direction in the syringe in any suitable manner. Exemplary drive mechanisms in accordance with the disclosed subject matter are described with reference to FIGS. 6-9.

In use, a user may remove a cover (not shown) disposed proximate the bottom surface of the wearable automatic injection device 500 to expose adhesive surface 516 and aperture 518 and place the wearable automatic injection device 500 with aperture 518 proximate the injection site. The user may then actuate the actuation button 510 of the wearable automatic injection device 500 when the sensor 514 detects contact with the injection site. The injection needle may then be urged into an extended position extending through the hole 518 to penetrate the injection site. In addition, if the sensor no longer detects contact with the injection site, the injection needle may be retracted and delivery of the therapeutic agent may stop. At the same time or in close temporal proximity to the injection needle being urged into an extended position, the fluid actuation mechanism may move the proximal stopper with the dispensing needle in a distal direction toward the distal stopper to penetrate the distal stopper and allow the therapeutic agent in the prefilled container to flow through the dispensing needle as the distal stopper moves distally to the distal most end of the prefilled container to force the therapeutic agent out of the syringe and into the fluid pathway via the dispensing needle. The therapeutic agent may thus flow through the dispensing needle, through the proximal stopper and into the tubing. The therapeutic agent may continue to flow through the tubing through the proximal end of the prefilled container to the injection needle to deliver the therapeutic agent to the patient. In this manner, and as described further herein, the sterile conditions of the prefilled container and injection assembly are maintained.

FIG. 6 shows an exemplary fluid drive mechanism configured as a hydraulic actuated drive mechanism 600. With reference to FIG. 6, the hydraulic actuated drive mechanism may include a movable member, which can be configured, for example and without limitation, as a piston assembly 602 or an expandable bellows assembly 604. As shown in FIG. 6 the piston assembly 602 or bellows assembly 604 may move telescopically or otherwise expand from a retracted position to an extended positon by injection of a hydraulic fluid, such as mineral oil, water, synthetic oil or any other suitable hydraulic fluid. The hydraulic fluid may be pressurized in any suitable manner, including via a mechanical energy source, such as a compression spring 606 and/or a motor driven by an electric power source, such as a battery. The hydraulic fluid may be delivered via hydraulic fluid tubing 614 to engage a first surface of the piston assembly 602 or bellows assembly 604. A second surface of the piston or bellows assembly 602, 604 may be coupled to a portion of the proximal stopper and may urge the proximal stopper and dispensing needle toward the distal stopper. In the manner, the contents of the syringe may flow through the dispensing needle, tubing, and injection needle via the proximal end of the syringe. In some examples, the movable member may include a flexible shaft having a tubular member and rigid beads over-molded on the tubular member.

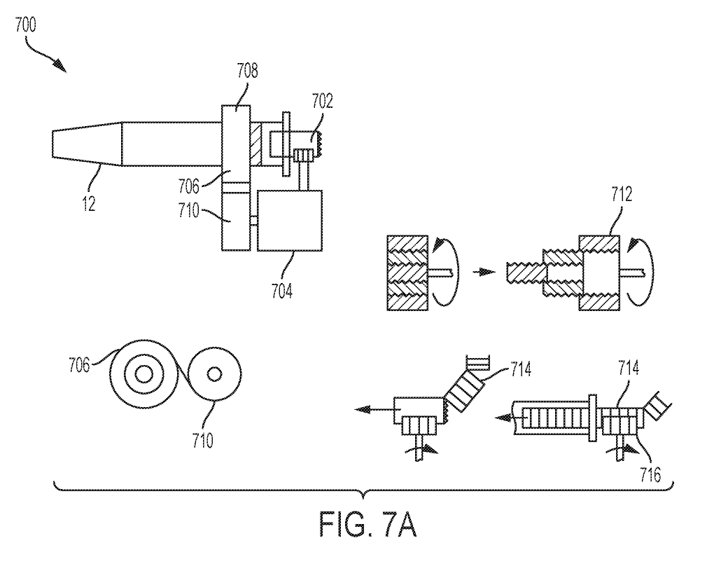

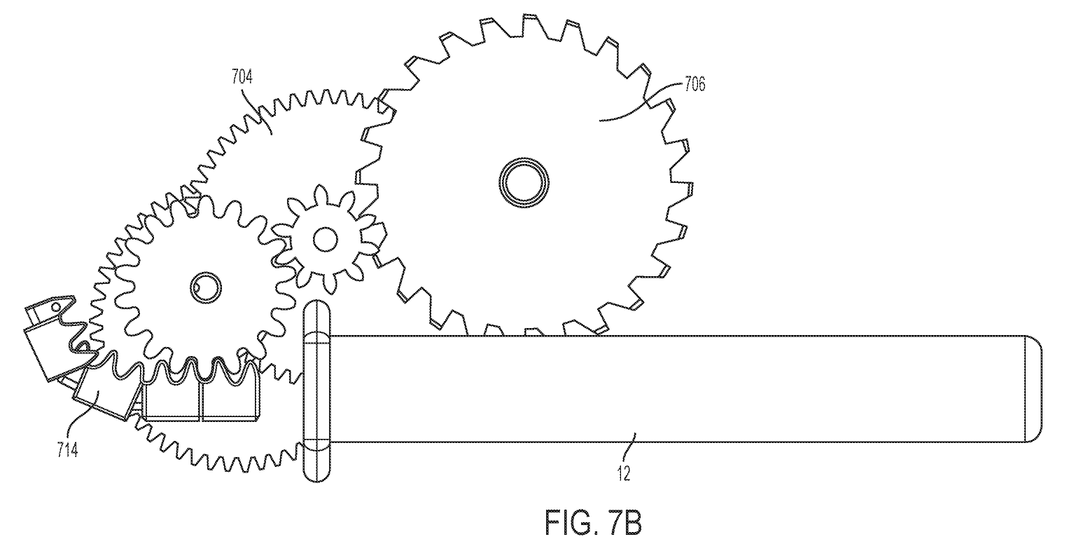

FIGS. 7A and 7B together show an exemplary fluid drive mechanism configured as a spring motor mechanism assembly 700. With reference to FIGS. 7A and 7B, the proximal and distal stoppers may be urged in the distal direction within the syringe to deliver a therapeutic agent through the proximal end of the syringe to an injection needle via a spring motor mechanism assembly 700. The spring motor mechanism assembly 700 may include a piston 702, which can be configured as a telescopic or articulated piston, gear train 704, drums 706 and 710 and motor 708. A constant force spring may be wound around drums 706 and 710. Upon actuation of the fluid drive mechanism, the drums 706, 710 may rotate and apply torque through a gear train 704 to move the piston 702 via a linear actuator, which can be configured for example and without limitation as a lead screw 712 or an articulated rack 714 and pinion 716. In another example, gear train 704 may be powered by a power source such as a battery-powered electric motor. In another example, the gear train 704 may be powered by a clock spring.

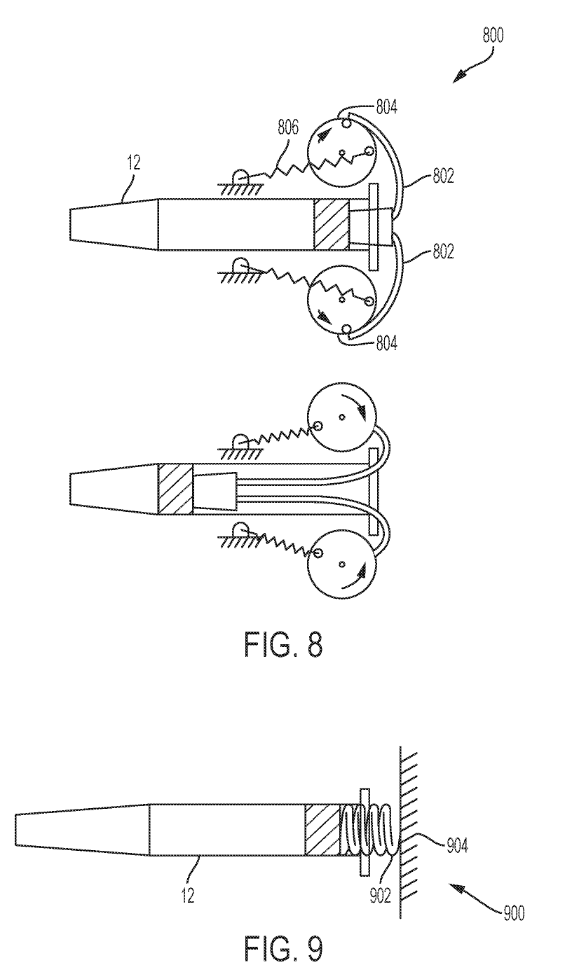

FIG. 8 shows an exemplary fluid drive mechanism configured as a flat spring mechanism assembly 800. With reference to FIG. 8, the proximal and distal stoppers may be urged in the distal direction within the syringe to deliver a therapeutic agent through the proximal end of the syringe to an injection needle via a flat spring mechanism assembly 800. The flat spring mechanism assembly 800 may be driven by a cam 804. The assembly 800 may include at least one flat spring 802, at least one cam 804 and spring 806. As embodied herein, two flat springs 802 each joined to the proximal stopper proximate a first end may be connected to a corresponding cam 804 proximate an opposing end. Upon actuation of the fluid drive mechanism and rotation of the cams 804, each flat spring 802 may be configured to pivot through the proximal end of the syringe to urge the proximal stopper toward the distal end of the syringe to deliver the therapeutic agent. In another example, the at least one cam 804 may be powered by a power source such as a battery.

FIG. 9 shows an exemplary fluid drive mechanism configured as a compression spring mechanism assembly 900. With reference to FIG. 9, the proximal and distal stoppers may be urged in the distal direction within the syringe to deliver a therapeutic agent through the proximal end of the syringe to an injection needle via a compression spring mechanism 900. The compression spring mechanism assembly 900 may be driven by a cam (not shown). The assembly 900 may include a compression spring 902 joined proximate a first end to the proximal stopper and proximate an opposing end to a rigid surface 904. Upon actuation of the fluid drive mechanism, the compression spring may expand through the proximal end of the syringe to urge the proximal stopper toward the distal end of the syringe to deliver the therapeutic agent. In another example, the compression spring 902 may be powered by a power source such as battery.

FIG. 10 shows an exemplary needle insertion and retraction system 1000 in accordance with the disclosed subject matter. With reference to FIG. 10, the needle insertion and retraction system 1000 may include a linkage 1002, which can be configured as a four-bar linkage, biased by spring 1006 to retain linkage 1002 in a first positon with injection needle 1004 in a retracted position and urge linkage 1002 to a second position with injection needle 1004 in an extended position for injection. Continuation of the motion of the linkage 1002 may urge the injection needle 1004 from the extended position to the retracted position. In another example, the needle insertion and/or retraction may be powered by a power source such as battery.

FIG. 11 shows a flow diagram 1100 of an exemplary sterilization process for a prefilled container and injection assembly, such as for prefilled container and injection assembly 10, for assembling a container prefilled with therapeutic agent with a sterile fluid pathway in an aseptic filling area. With reference to FIG. 11, Step 1110 includes providing a prefilled container, such as syringe 12, having a sterile barrier proximate the distal end 14. For example and as embodied herein, the sterile barrier can be configured as a rigid needle shield or sealing member disposed about the tip of distal end 14. Step 1120 includes joining a fluid path with the syringe in a cleanroom to form a prefilled container and injection assembly. In step 1120, a sterile barrier 329 is joined proximate a distal end of the injection needle 328. The prefilled container and injection assembly is then packed in a packing container 11. Step 1130 includes low temperature sterilization (e.g. room temperature, about 18-25° C.) of the container 11 having the prefilled container and injection assembly disposed therein. Step 1140 includes removing the prefilled container and injection assembly from packing container 11 in a cleanroom assembly area, inserting the prefilled container and injection assembly into the wearable automatic injection device housing 502 in the cleanroom assembly area and joining adhesive layer 516 to a bottom surface of the housing 502 to form a wearable automatic injection device 500. Step 1150 includes delivering the packing container 11 with device 500 to the user, and Step 1160 includes removing device 500 from packing container 11, removing the adhesive liner 516 from device 500, and removing the sterile barrier 329 from the patient needle 328 by the user prior to use of the device 500.

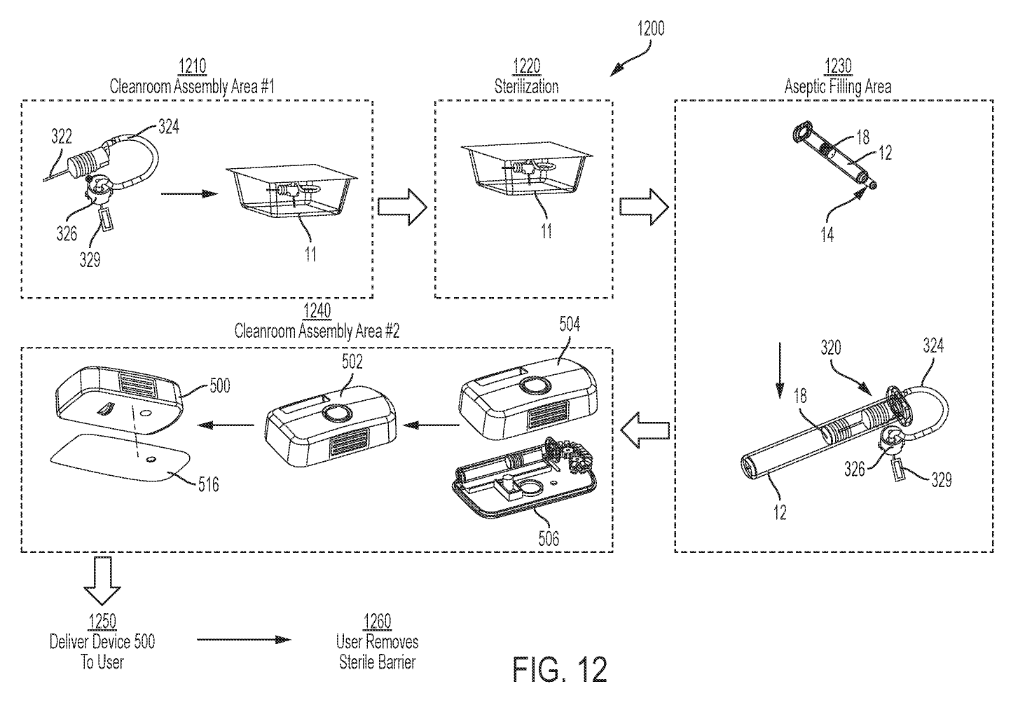

FIG. 12 shows a flow diagram 1200 of another exemplary sterilization process for a prefilled container and injection assembly, such as for prefilled container and injection assembly 300 for assembling a container prefilled with therapeutic agent in an aseptic filling area and then re-lidding the prefilled container in an aseptic tray and moving to an aseptic assembly area to finish the assembly. Step 1210 includes assembling, in a cleanroom, a fluid pathway subassembly having a proximal stopper 320, penetrating member 322, and tubing 324 coupled to needle hub 326 and injection needle 328. The injection needle 328 is covered by sterile barrier 329, as described herein. The fluid pathway subassembly is placed in a packing container 11. In Step 1220, the packing container 11 including the fluid pathway subassembly is sterilized. Step 1230 includes providing a container, such as syringe 12, in an aseptic area, Syringe 12 may have a sterile barrier disposed proximate a distal end 14 thereof. In the aseptic area, the syringe 12 may be filled (e.g. with therapeutic agent) and a stopper 18 inserted therein. The proximal stopper 320 of the fluid pathway subassembly may be coupled to the filled syringe 12 to form a prefilled container and injection assembly. Step 1240 includes coupling the prefilled container and injection assembly to the wearable automatic injection device housing 502 in a cleanroom and joining adhesive layer 516 to the bottom surface of the housing 502 to form a wearable automatic injection device 500. Step 1250 includes delivering the packing container 11 with device 500 to the user, and Step 1260 includes removing device 500 from packing container 11, removing the adhesive liner 516 from device 500, and removing the sterile barrier 329 from the patient needle 328 by the user prior to use of the device 500.

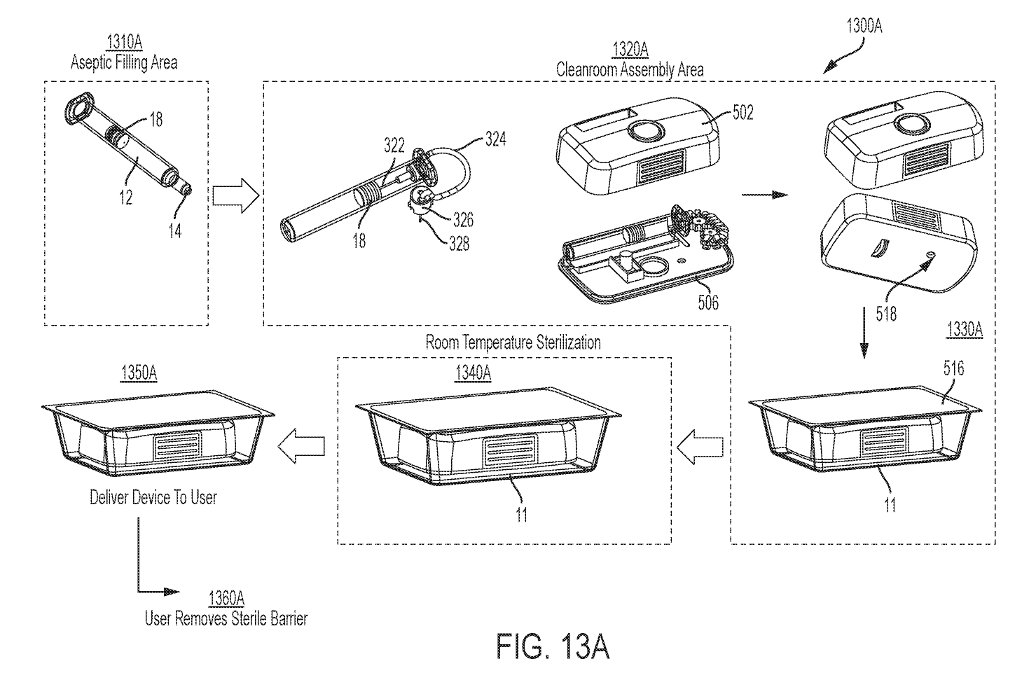

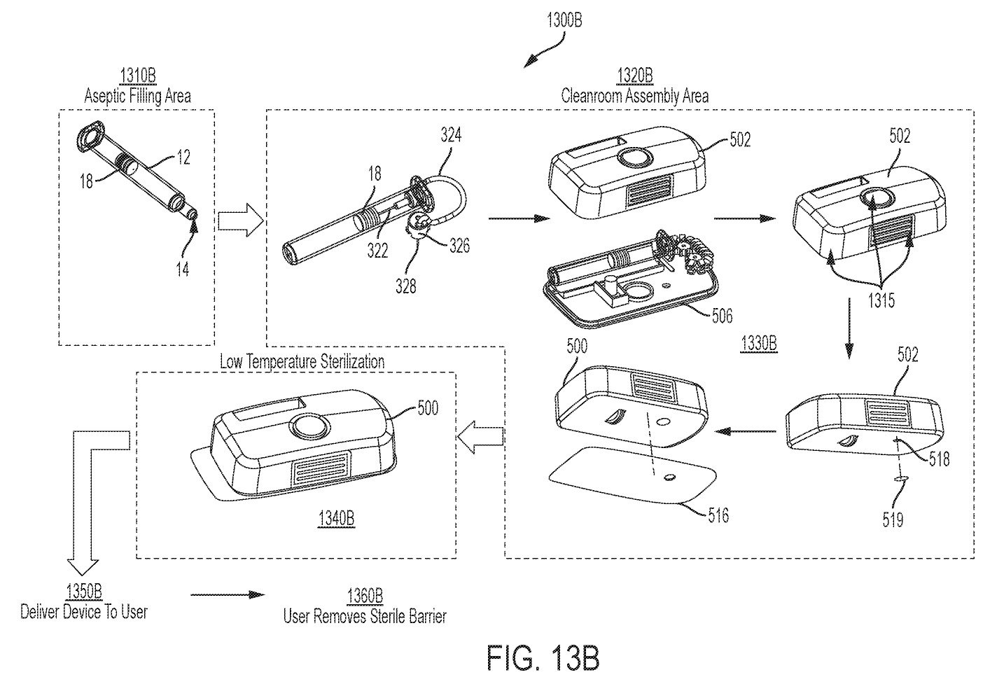

FIG. 13A shows a flow diagram 1300A of another exemplary sterilization process for a prefilled container and injection assembly, such as for prefilled container and injection assembly 300 for assembling a container prefilled with therapeutic agent in an aseptic filling area and coupling the prefilled container with a fluid pathway subassembly and subjecting the assembly to a room temperature sterilization process. Step 1310A includes filling a container, such as syringe 12, through a proximal end thereof, the syringe 12 having a sterile barrier disposed proximate a distal end 14 thereof, and inserting a stopper 18 through the proximal end of syringe 12. Step 1320A includes coupling the prefilled syringe 12 to a fluid pathway subassembly in a cleanroom assembly area, as described herein, to form a prefilled container and injection assembly. The prefilled container and injection assembly is placed in a wearable automatic injection device housing 502 to form a wearable automatic injection device 500. Step 1330A includes placing the assembled device 500 in a packing container 11, and Step 1340A includes subjecting the entire packing container 11 with the device 500 disposed therein to room temperature sterilization (e.g., using NO₂). Step 1350A includes delivering the packing container 11 including device 500 to the user, and Step 1360A includes removing device 500 from packing container 11, removing the adhesive liner 516 from device 500, and removing the sterile barrier 329 from the patient needle 328 by the user prior to use of the device 500.

FIG. 13B shows a flow diagram 1300B of another exemplary sterilization process for a prefilled container and injection assembly, such as for prefilled container and injection assembly 300, for assembling a container that is prefilled with therapeutic agent in an aseptic filling area and coupling the prefilled container into a fluid pathway subassembly to form a prefilled container and injection assembly, and subjecting the prefilled container and injection assembly to a room temperature sterilization process. Step 1310B includes filling a container, such as syringe 12, through a proximal end thereof, the syringe 12 having a sterile barrier disposed proximate distal end 14 thereof, and inserting a stopper 18 through the proximal end of syringe 12. Step 1320B includes coupling the prefilled syringe 12 to a fluid pathway assembly in a cleanroom assembly area to form a prefilled container and injection assembly. The prefilled container and injection assembly is placed in a wearable automatic injection device housing 502. The housing 502 top and bottom surfaces, along with any openings in housing 502, are hermetically sealed at 1315. Step 1330B includes placing a sterile barrier proximate the aperture 518 and joining adhesive layer 516 to the bottom surface of the housing 502 to form a wearable automatic injection device 500. Step 1340B includes low temperature sterilization of the device 500. Step 1350A includes delivering the device 500 to the user, and Step 1360B includes removing the adhesive liner 516 from device 500, and removing the sterile barrier 329 from the patient needle 328 by the user prior to use of the device 500.

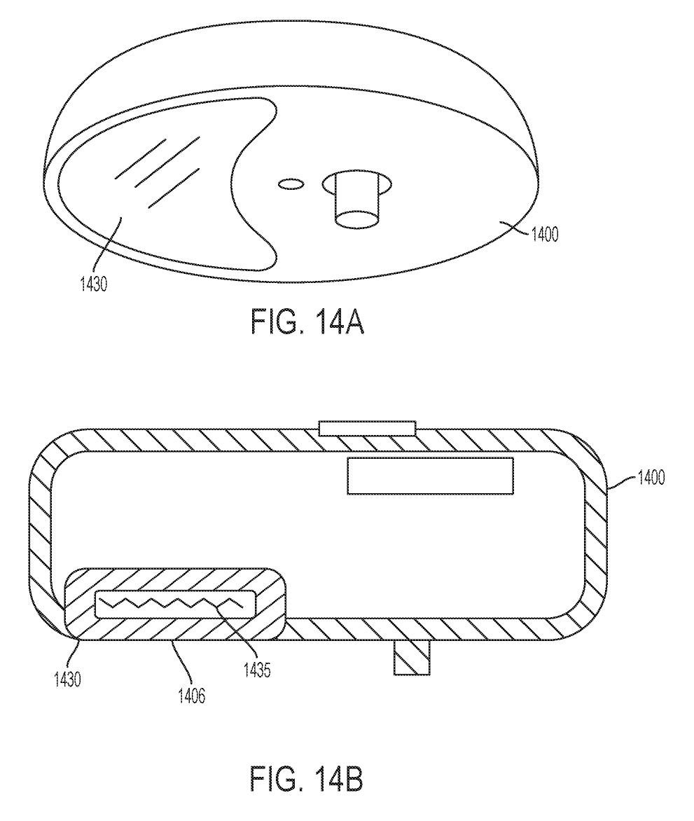

FIGS. 14A and 14B together illustrate another embodiment of a wearable automatic injection device 1400, having a similar configuration as the wearable injection device 500 shown in FIGS. 5A-5C, and including pain reduction components. With reference to FIG. 14A, the wearable automatic injection device 1400 may include a high thermal mass member 1430 disposed proximate a bottom surface of the device 1400. The high thermal mass member 1430 can have any suitable shape and size, include a material of thermal mass 1406, and can be manufactured using any suitable materials such as stainless steel, aluminum, copper, and/or other materials having thermally effusive properties. The high thermal mass member 1430 can thus be configured to contact an injection site, such as a patient's skin. The high thermal mass member 1430 can be configured to reach thermal equilibrium when the wearable automatic injection device 1400 is stored at cold temperatures (e.g., in a refrigerator). Upon removal of the device 1400 from a refrigerated environment, the device 1400 can be placed proximate the injection site on the patient's skin, and the high thermal mass member 1430 can reduce the temperature of and around the injection site on the patient's skin to provide a numbing sensation to reduce the sensation of pain by the patient upon insertion of the needle. Additionally or alternatively, the high thermal mass member 1430 may utilize a phase-change material 1435 internal to its construction in addition to the conductive and effusive contact surface to reduce the temperature ramp rate of the high thermal mass member 1430. The phase change material may be manufactured using any suitable material(s) such as stainless steel, aluminum, and or copper. A melting point of the phase-change material can be adjusted to maintain the high thermal mass member 1430 at a predetermined cool temperature for a longer duration. In some examples, the phase-change material may be removable from the device and may be kept in a refrigerated environment prior to insertion into the device. The melting point range for the phase change material may be between about 5° C. (typical refrigerator temperature) and about 10° C.

Additionally or alternatively, a thermal liner can be disposed proximate an adhesive layer on the bottom surface of the device 1400 and can be configured to adhere to an injection site, such as a patient's skin. Such a thermal liner may be manufactured using one or more materials having insulating properties similar to the high thermal mass member 1430 and thus configured to reduce the temperature ramp rate after removal of the device 1400 from a refrigerated environment and prior to application to the patient's skin. When the adhesive layer is removed, the high thermal mass member 1430 may be exposed and can be positioned against the patient skin when the device 1400 is on the injection site. Additionally or alternatively, the device 500 may include other pain reduction components, such as a vibrating component configured to vibrate the device during needle insertion to distract needle insertion sensation.

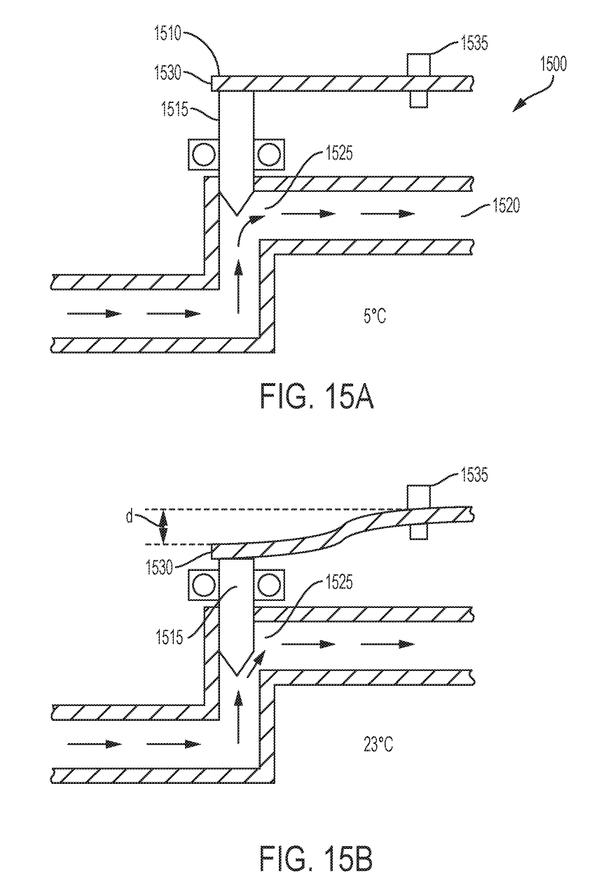

FIGS. 15A and 15B together illustrate an exemplary flow regulator 1500 for use in a wearable automatic injection device, such as device 500. The flow regulator 1500 can be used to provide a substantially constant fluid flow rate across the operating temperature range of the device to administer the therapeutic agent at a substantially uniform flow rate. In some examples, the flow regulator 1500 may be used to control greater and lesser rates of flow during administration of the therapeutic agent. The flow regulator 1500 may include an actuator 1510, which can include materials having different coefficients of thermal expansion. In some examples, the actuator 1510 may include a bimetallic structure, such as copper and steel joined together in the shape of a beam, coil, disc or any other suitable shape. The actuator 1510 may be joined to a needle valve 1515, which may be disposed within the fluid path 1520 of a wearable automatic injection device, such as device 500. The actuator 1510 can be configured to utilize the different coefficients of thermal expansion (e.g. of the two metals in the bimetallic structure) to expand or bend as temperature changes to urge the needle valve 1515 into the fluid path 1520 and partially obstruct an orifice 1525 in the fluid path 1520, thereby decreasing the rate of fluid flow through the fluid path 1520 due to the smaller size of the orifice 1525. The geometry of the actuator 1510 and needle valve 1515 may be configured such that the size of the orifice 1525 can be precisely controlled at about 5° C. (common refrigerator temperature), as shown in FIG. 15A, and at about 23° C. (typical room temperature), as shown in FIG. 15B, to achieve the same fluid flow rate across the operating temperature range of the device. The size (e.g. diameter) of the orifice may reduce by at room temperature based on the effect of temperature on the viscosity of the therapeutic agent to be delivered by the device 500. In some examples, the size of the orifice may be reduced by about 5% to about 65%, in other examples, the reduction may be 15% to about 45%, in some examples the reduction may be at about 20% to about 40%. The actuator 1510 may have a fixed end 1535 opposite a free end 1530 joined to the needle valve 1515. As shown in FIG. 15A, at a first temperature of about 5° C., the fixed end 1535 and the free end 1530 may be substantially parallel. As shown in FIG. 15B, at a second temperature of about 23° C. and as compared to the first temperature, the free end 1530 of the actuator 1510 may move a distance “d” relative to the fixed end 1535.

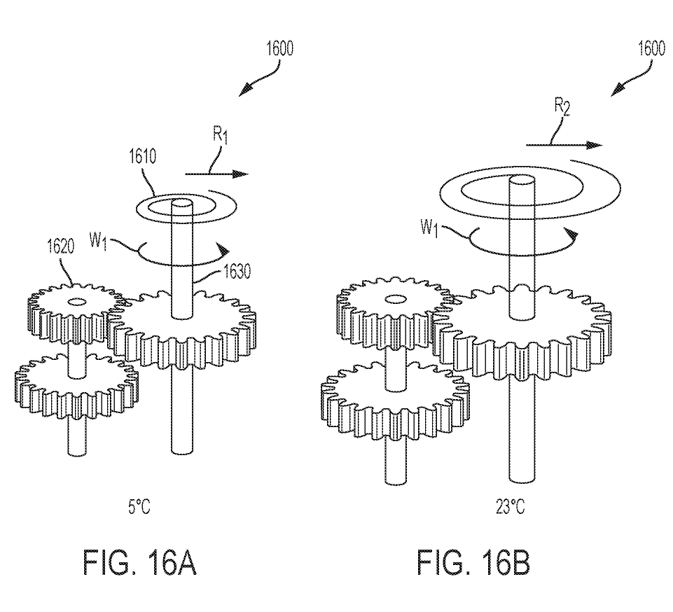

FIGS. 16A and 16B together illustrate another exemplary embodiment of a flow regulator 1600 for use in a wearable automatic injection device, such as device 500. The flow regulator 1600 may include an actuator component 1610, which can include materials having different coefficients of thermal expansion. In some examples, the actuator component 1610 may include a bimetallic structure, such as copper and steel joined together to form a suitable shape. In some examples, the bimetallic structure may have the shape of a coil or ring. The bimetallic structure may be joined to a rotating shaft 1630 inside the device, e.g. device 500. The rotating shaft 1630 can be driven by a gear train 1620 powered by a power source, such as a clock spring, a compression spring or other suitable power source. The bimetallic element of the actuator component 1610 may include a low-mass component configured as a wind vane in a speed governor mechanism. For example, and as embodied herein, the wind vane can be configured to rotate at speeds of about 3000 RPM or greater via a gear ratio of 2000:1 or greater. The speed governor mechanism may utilize air resistance to control the rotational speed of the gear train 1620 and the injection rate. The structure of the actuator component 1610 having the bimetallic element may be configured to utilize the different coefficients of thermal expansion of the two metals to expand as the operating temperature increases, thereby causing the diameter of the speed governor to increase, increasing the air resistance encountered by the speed governor and reducing the rotational speed of the gear train 1620. With the gear train 1620 rotating at a reduced speed, the injection rate can similarly be reduced. The geometry of the actuator 1610 having the bimetallic element can be configured such that the diameter of the governor can be precisely controlled at about 5° C. (common refrigerator temperature) to rotate at a relatively faster angular velocity and at about 23° C. (typical room temperature) to rotate at a relatively slower angular velocity to achieve the same fluid flow rate across the operating temperature range of the device.

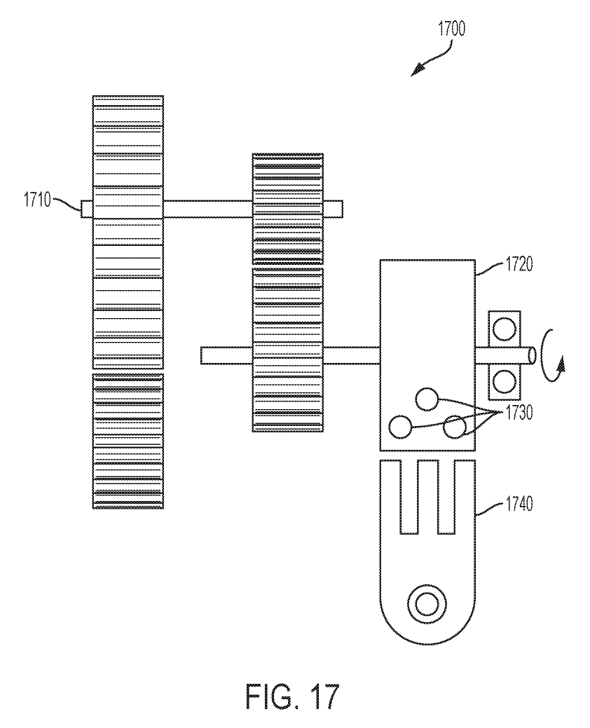

FIG. 17 illustrates an exemplary audible feedback mechanism for use in a wearable automatic injection device, such as device 500. The audible feedback mechanism 1700 can be configured to generate an audible tone to provide feedback to a user of the device, for example, to indicate start of or completion of the injection. The audible feedback mechanism 1700 can include a gear train 1710 having one or more gear parts. The gear train 1710 can be joined to a rotating drum 1720 having one or more pins 1730 disposed therein, and the rotating drum 1720 may be joined to a comb or fork 1740. One or more of the components of the audible feedback mechanism 1700 may be manufactured using metal, plastics, and/or any suitable materials. The rotating drum 1720 and/or gear train 1710 can include protruding pins or gear teeth, which can be configured to contact the teeth of the comb or fork 1740 at a predetermined time to generate high frequency vibration to create audible tones. The pins or gear teeth of gear train 1710 can be spaced apart from each other to engage the comb or fork 1740 in a predetermined, timed sequence to thereby generate a predetermined melody. Additionally or alternatively, the individual comb or fork teeth 1740 can be sized to generate tones of predetermined frequency as they vibrate. In some examples, the frequency of the vibration is between about to be between 500 Hz and 4000 Hz.

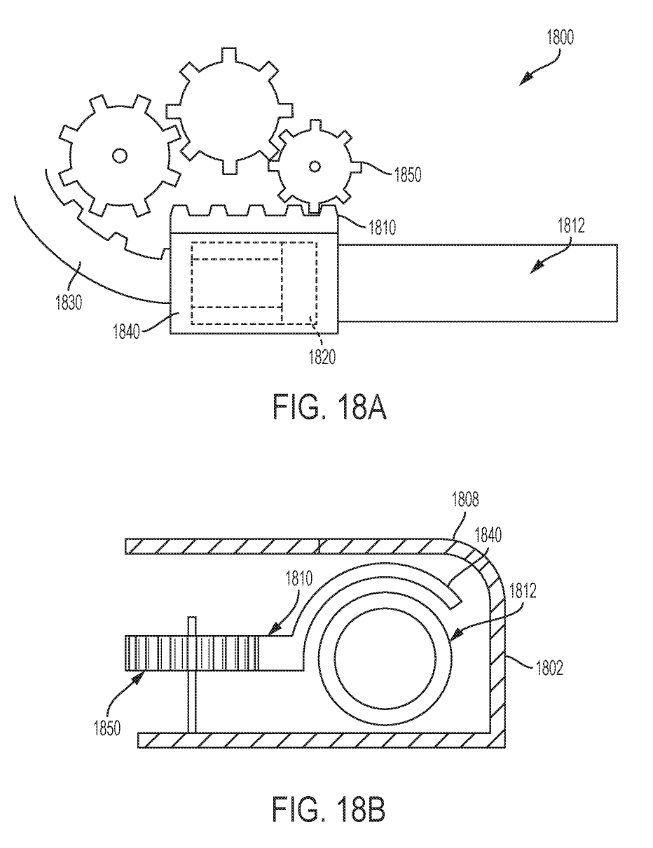

FIGS. 18A and 18B together illustrate an exemplary visual feedback mechanism 1800 for use in a wearable automatic injection device, such as device 500. With reference to FIGS. 18A and 18B, the visual feedback mechanism 1800 can include rack 1810 and pinion 1850 components to urge plunger rod 1830 of the device and injector sleeve 1840 together as the plunger rod 1830 urges stopper 1820 within the therapeutic agent container 1812. In this manner, a user may view the drug container 1812 and its contents through a viewing window 1808 disposed in the housing 1802 of the device. The sleeve 1840 can include a suitable visual indicator, such as a distinct color, pattern, text, or image, to allow visual indication of the movement of the plunger.

In another example, the wearable automatic injection device, such as device 500, may include a mechanical visual indicator to provide feedback to a user of the device status. The mechanical visual indicator may provide a visual indication to the user that the wearable automatic injection device is in a “ready” configuration e.g. when the device is adequately adhered to the patient skin and the injection can be initiated. A viewing window can be disposed in the housing of the device. A mechanism actuated by a skin sensor (such as sensor 514 described herein) can toggle a visual indicator, for example and as embodied herein, indicating “no go” (or a red color or particular symbol) when the sensor does not detect contact with the injection site and indicating “go” (or a green color or other particular symbol) when the sensor detects contact with the injection site. Additionally or alternatively, an indicator can be urged into alignment with the viewing window after completion of a successful injection and/or an interrupted injection by joining the indicator mechanism to the prefilled container and injection assembly.

In another example, a wearable automatic injection device, such as device 500, may include a two-stage actuation button in combination with a viewing window to allow a user to prime the fluid pathway and/or purge air from the fluid pathway prior to delivery of the therapeutic agent. In this manner, priming the fluid pathway and/or purging air from the fluid pathway can include tilting the device about 90 degrees to a vertical orientation prior to adhering the device to the injection site in a horizontal orientation. In the vertical orientation, the user can actuate the two-stage actuation button to a first position, which opens a viewing window to visually inspect the syringe therethrough, positions an air bubble inside the prefilled syringe, and initiates movement of the proximal stopper and the dispensing needle toward the distal stopper. In this manner, the dispensing needle can penetrate the distal stopper, thereby priming the fluid pathway and/or purging air from the prefilled syringe prior to adhering the device to the skin. The user can place the primed device proximate the injection site, and actuate the two-stage actuation button to a second position, or alternatively, actuate a second actuation button, to deliver the therapeutic agent, as described herein, without air bubbles.

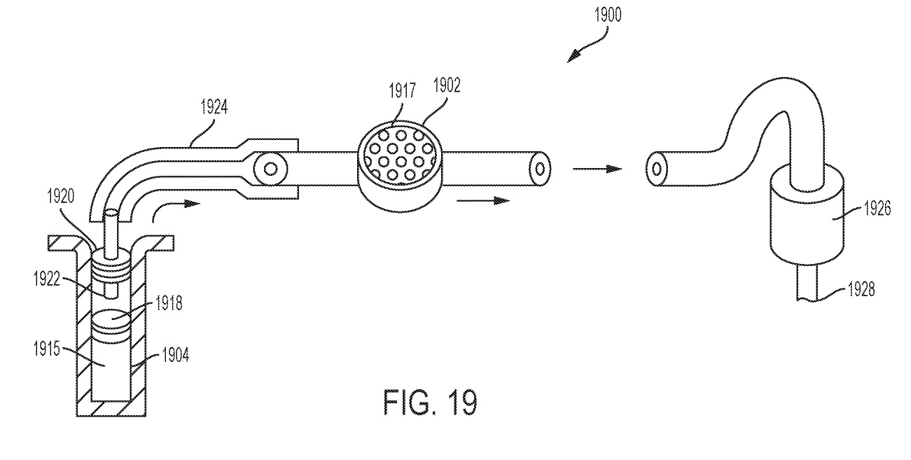

FIG. 19 illustrates an exemplary reconstitution assembly 1900 of a wearable autoinjector device, such as device 500. The reconstitution assembly 1900 can be used to reconstitute one or more therapeutic agents in a dried (e.g. lyophilized) or partially dried form with a mixing liquid. The mixing liquid 1915 may reconstitute the dried or partially dried therapeutic agent, and the reconstituted therapeutic agent may be delivered to a patient via subcutaneous injection, as described herein. With reference to FIG. 19, the reconstitution assembly 1900 may include a container 1904 such as a syringe, cartridge, vial, ampule, or other suitable container to contain a suitable mixing fluid 1915, such as water. Similar to the prefilled container and injection assembly 300 described herein, the container 1904 can include a first and second stopper such as distal stopper 1918 that can be pierced by a dispensing needle 1922 joined to a proximal stopper 1920. The proximal stopper 1920 is in fluid communication via a sterile path 1924 with a reconstitution chamber 1902. The reconstitution chamber 1902 can include one or more internal chambers for holding the dried or partially dried therapeutic agent 1917 and mixing the therapeutic agent 1917 with the mixing fluid 1915 to reconstitute the therapeutic agent into a fluid form. The fluid form of the therapeutic agent may flow thorough another portion of the sterile path 1924 to a needle hub 1926 joined to an injection needle 1928 for delivery to the patient via subcutaneous injection.

In use, as the therapeutic agent delivery is initiated in the device, the first stopper 1918 can be pierced by the dispensing needle 1922 joined to the second stopper 1920, and the mixing fluid 1915 in the container 1904 can be urged through the sterile fluid path 1924 to the mixing chamber 1902 to be mixed with the dried therapeutic agent to create a fluid therapeutic agent solution. The flow of the mixing fluid under pressure can mix the dried therapeutic agent with the mixing fluid. In some examples, the mixing chamber 1902 can include a separate power source configured to power a mixing mechanism (not shown) to provide additional or alternative mixing of the dried therapeutic agent. The geometry of the mixing chamber 1902 can be such that the therapeutic agent is sufficiently mixed without damage to the therapeutic agent molecules prior to being urged to the distal end of the sterile fluid path 1924 for delivery to the patient via injection needle 1928. In some examples, the mixing chamber 1902 may include separate internal chambers for holding two or more separate therapeutic agents and/or separate amounts of dried or partially dried therapeutic agents. The sterile path 1924 may deliver the mixing fluid 1915 to each of the internal chambers to allow the therapeutic agents to be mixed and reconstituted together, or alternatively, separately reconstituted and then delivered to the patient, e.g. via a bifurcated portion of the sterile path 1924. The reconstitution assembly 1900 may be contained in a wearable automatic injection device housing, such as housing 502 of device 500.

In another example, a wearable automatic injection device, such as device 500, the device may include an electronic circuit board to provide electronic features for the device. For example, and as embodied herein, the electronic circuit board can provide a power and/or data connection from the wearable automatic injection device to an external device, such as a personal computer or mobile device, via a data bus and power connector, such as USB, micro USB, Lightning, or other standard or proprietary data bus and/or power connector. In this manner, the device can obtain power for an electric motor for use instead of, or in addition to, the fluid drive mechanisms described herein. Additionally or alternatively, the data bus and power connection can also provide data collection, such as telemetry data, from the device 500 to the personal computer or mobile device. In addition, or as a further alternative, the data bus and power connection can also allow the external device to provide a user interface for control of the wearable automatic injection device and/or to provide visual or audible feedback to the user in addition to or as an alternative to the mechanical control and feedback features described herein.

Exemplary automatic injection devices may be used to administer essentially any substance or therapeutic agent that is suitable for administration by injection. Typically, the substance or therapeutic agent will be in a fluid, e.g., liquid form, although medications in other forms such as gels or semi-solids, slurries, particulate solutions, etc. also may suitable for use with the wearable automatic injection devices according to the disclosed subject matter. Preferred therapeutic agents include biological agents, such as antibodies, cytokines, vaccines, fusion proteins and growth factors.

The contents of all references, including patents and patent applications, cited throughout this application are hereby incorporated herein by reference in their entirety. The appropriate components and methods of those references may be selected for the invention and embodiments thereof. Still further, the components and methods identified in the Background section are integral to this disclosure and can be used in conjunction with or substituted for components and methods described elsewhere in the disclosure within the scope of the invention.

In describing exemplary embodiments, specific terminology is used for the sake of clarity. For purposes of description, each specific term is intended to at least include all technical and functional equivalents that operate in a similar manner to accomplish a similar purpose. Additionally, in some instances where a particular exemplary embodiment includes a plurality of system elements or method steps, those elements or steps may be replaced with a single element or step. Likewise, a single element or step may be replaced with a plurality of elements or steps that serve the same purpose. Further, where parameters for various properties are specified herein for exemplary embodiments, those parameters may be adjusted up or down by 1/20th, 1/10th, ⅕th, ⅓rd, ½, etc., or by rounded-off approximations thereof, unless otherwise specified. Moreover, while exemplary embodiments have been shown and described with references to particular embodiments thereof, those of ordinary skill in the art will understand that various substitutions and alterations in form and details may be made therein without departing from the scope of the invention. Further still, other aspects, functions and advantages are also within the scope of the invention.

Exemplary flow diagrams are provided herein for illustrative purposes and are non-limiting examples of methods. One of ordinary skill in the art will recognize that exemplary methods may include more or fewer steps than those illustrated in the exemplary flow diagrams, and that the steps in the exemplary flow diagrams may be performed in a different order than shown.

Other embodiments of the disclosure will be apparent to those skilled in the art from consideration of the specification and practice of the disclosure herein. It is intended that the specification and examples be considered as exemplary only, with a true scope and spirit of the invention being indicated by the following claims.

Various embodiments of wearable automatic injection devices have been described herein. These embodiments are given by way of example and are not intended to limit the scope of the present invention. It should be appreciated, moreover, that the various features of the embodiments that have been described may be combined in various ways to produce numerous additional embodiments. Moreover, while various materials, dimensions, shapes, injection locations, etc. have been described for use with disclosed embodiments, others besides those disclosed may be utilized without exceeding the scope of the invention.

What is claimed is:

1. A wearable automatic injection device for providing an injection of a therapeutic agent into a patient, the wearable automatic injection device comprising:
a housing comprising a contact portion securable to the patient;
an injection assembly moveably disposed in the housing holding an injection needle for insertion into the patient, the injection assembly movable between a retracted position in which the injection needle does not protrude outside the housing and an extended position in which the injection needle protrudes outside the housing; and
a prefilled container assembly disposed in the housing for holding the therapeutic agent, the prefilled container assembly comprising a barrel with a sealed distal end, the barrel having a first stopper, a second stopper spaced from and movable relative to the first stopper, and a dispensing member coupled to a distal portion of the second stopper and configured to engage the first stopper,
wherein the dispensing member is in fluid communication with the injection needle.

2. The wearable automatic injection device of claim 1, further comprising a fluid line coupled to and extending between the injection needle and the dispensing member.

3. The wearable automatic injection device of claim 2, wherein the fluid line is movable with the injection assembly.

4. The wearable automatic injection device of claim 1, further comprising a fluid drive mechanism configured to urge the second stopper in a distal direction toward the first stopper.

5. The wearable automatic injection device of claim 4, wherein the fluid drive mechanism is selected from the group consisting of a: hydraulic mechanism, pneumatic mechanism, spring motor or clock spring mechanism, cam actuator, and compression spring.

6. The wearable automatic injection device of claim 1, wherein the housing has a top surface comprising a viewing window radially aligned with the contents of the barrel.

7. The wearable automatic injection device of claim 1, wherein the housing has a bottom surface comprising an adhesive surface defining the contact portion, the bottom surface having an aperture therethrough, the injection needle extending through the aperture when the injection assembly is in the extended position.

8. A prefilled container assembly comprising:
a container including a barrel having a sealed distal end and a proximal end;
a first stopper and a second stopper spaced from the first stopper toward the proximal end and movable relative to the first stopper;
a dispensing member coupled to a distal portion of the second stopper and configured to engage the first stopper; and
a fluid tube in fluid communication with the dispensing member and an injection needle.

9. The prefilled container assembly of claim 8, wherein the container comprises a syringe or a cartridge.

10. A method of delivering a therapeutic agent to a patient comprising:
providing a container prefilled with the therapeutic agent and a first stopper;
urging a second stopper having a dispensing needle extending therefrom into engagement with the first stopper; and
delivering the therapeutic agent to the patient via an injection needle in fluid communication with the second stopper.

11. A fluid delivery device comprising:
a housing having a closed distal end and a proximal end opposite the distal end;
a first stopper spaced from the distal end and defining a fluid reservoir therebetween;
a second stopper spaced from the first stopper toward the proximal end; and
a fluid line having a first end coupled to the second stopper and a second end coupled to an injection needle.

12. A method of assembling a prefilled container and injection assembly comprising:
sealing a distal end of a container;
inserting a fluid through a proximal end of the container;
inserting a first stopper through the proximal end of the container to seal the fluid therein;
inserting a second stopper through the proximal end of the container, the second stopper having a dispensing member extending therefrom toward the first stopper,
joining the second stopper to a first end of a tubing member extending through the proximal end of the container; and
joining an opposing end of the tubing member to an injection needle.

13. A wearable automatic injection device for providing an injection of a therapeutic agent into a patient, the wearable automatic injection device comprising:
a housing comprising a contact portion configured to secure to the patient;
an injection assembly moveably disposed in the housing, the injection assembly including an injection needle to be inserted into the patient, the injection assembly movable between a retracted position in which the injection needle does not protrude outside the housing and an extended position in which the injection needle protrudes outside the housing;
a prefilled container assembly provided in the housing, the prefilled container assembly comprising a container with a sealed distal end, a first stopper spaced from the distal end and defining a fluid reservoir therebetween to hold a mixing fluid, a second stopper spaced from the first stopper toward the proximal end and movable relative to the first stopper, and a dispensing member extending from the second stopper toward the first stopper and configured to engage the first stopper, the dispensing member in fluid communication with the injection needle via a fluid line; and
a mixing chamber containing the therapeutic agent, the mixing chamber disposed between and in fluid communication with the prefilled container assembly and the injection needle via the fluid line.

14. A prefilled container assembly comprising:
a barrel having a sealed distal end and a proximal end;
a first stopper spaced from the distal end and defining a fluid reservoir therebetween;
a second stopper spaced from the first stopper toward the proximal end;
a dispensing member joined to the second stopper and configured to engage the first stopper; and
a fluid tube in fluid communication with the dispensing member and an injection needle.

15. The prefilled container assembly of claim 14, further comprising a fluid therapeutic agent in the fluid reservoir.

16. The prefilled container assembly of claim 15, wherein dispensing member is configured to engage the first stopper and define a channel therethrough to allow the fluid therapeutic agent to move from the fluid reservoir to the fluid tube through first and second stoppers.

17. The prefilled container assembly of claim 15, wherein the distal end of the barrel has a diameter less than a diameter of the proximal end of barrel.

18. The prefilled container assembly of claim 14, wherein the second stopper is configured to urge the first stopper toward the distal end of the barrel.

19. The prefilled container assembly of claim 15, wherein the injection needle is configured to penetrate the skin of a patient and deliver the fluid therapeutic agent from the fluid reservoir to patient.

20. The syringe assembly of claim 15, wherein the fluid therapeutic agent comprises a biological agent.
